(19) United States
(12) Patent Application Publication
(10) Pub. No.: US 2015/0045652 A1
(43) Pub. Date: Feb. 12, 2015

(54) Magnetic Resonance Apparatus with Optimized Arrangement of Local Coils

(71) Applicants: Yvonne Candidus, Furth (DE); Annemarie Hausotte, Erlangen (DE); René Kartmann, Nurnberg (DE); Ralf Ladebeck, Erlangen (DE); Daniel Paulus, Nurnberg (DE); Harald H. Quick, Erlangen (DE)

(72) Inventors: Yvonne Candidus, Furth (DE); Annemarie Hausotte, Erlangen (DE); René Kartmann, Nurnberg (DE); Ralf Ladebeck, Erlangen (DE); Daniel Paulus, Nurnberg (DE); Harald H. Quick, Erlangen (DE)

(21) Appl. No.: 14/456,748

(22) Filed: Aug. 11, 2014

(30) Foreign Application Priority Data
Aug. 12, 2013 (DE) 102013215902.1

(51) Int. Cl. G01R 33/34; A61B 5/055; A61B 5/00; G01R 33/48

(52) U.S. Cl. CPC G01R 33/34046; G01R 33/481; G01R 33/4812; A61B 5/055; A61B 5/0035
USPC 600/411; 324/322

(57) ABSTRACT

A magnetic resonance apparatus includes a patient couch with a couch surface on which a patient is supportable. In the patient couch, lower local coils for receiving excited magnetic resonance signals of the patient are arranged below the couch surface. On an upper side of the patient couch, connecting devices are arranged to sides. The magnetic resonance device includes upper local coils that are detachably connected mechanically and electrically with the connecting devices. The lower local coils and the upper local coils are connected to electronics that at least include a respective pre-amplifier and are connected via connection lines arranged outside the patient couch to an evaluation device. In a state in which the upper local coils are connected to the connecting devices, the upper local coils are fixed mechanically to the patient couch and cover the patient in a dome shape. The electronics are arranged in the patient couch.

This application claims the benefit of DE 10 2013 215 902.1, filed on Aug. 12, 2013, which is hereby incorporated by reference in its entirety.

### BACKGROUND

The present embodiments relate to a magnetic resonance apparatus.

The planning of treatment of patients with ionizing radiation may be undertaken based on CT images. During the acquisition of the CT images, the patient is to be arranged, if possible, in the same way as during the subsequent treatment. For example, the patient is arranged on a flat surface because the treatment table is likewise provided with a flat surface.

The CT images may be supplemented with magnetic resonance images. For example, magnetic resonance images exhibit a better contrast of soft tissue and provide additional functional information. For the registration of the MR images relative to the CT images, the patient may also be positioned on a flat surface during the acquisition of the MR images.

For the acquisition of MR images, radio frequency local coils have a dense fill level. For this purpose, upper local coils may be arranged on the patient. In the prior art, flexible upper local coils that may be laid directly on the patient may be used for this purpose. For planning the treatment of patients with ionizing radiation, the patient shape may be changed as little as possible by the imaging modality (e.g., by the upper local coils). In the prior art, it is therefore also known to arrange the upper local coils on special holders so that the upper local coils are spaced away from the patient. However, this leads to relatively voluminous arrangements.

If the magnetic resonance apparatus is to be combined with PET imaging, for the correct acquisition of patient data, as few hardware components is possible are arranged in the image field. Examples of such components are RF coils, cables, sheath current filters and more. If components are to be arranged in the image field, the components may be embodied as light and (PET)-transparent as possible. In order to enable an attenuation correction to be undertaken correctly, the location of the corresponding hardware components may be known. Therefore, as few movable or flexible components as possible may be used in the image field. This simplifies the attenuation correction of the hardware components arranged in the image field and leads to reproducible results during attenuation correction.

### SUMMARY AND DESCRIPTION

The scope of the present invention is defined solely by the appended claims and is not affected to any degree by the statements within this summary.

The present embodiments may obviate one or more of the drawbacks or limitations in the related art. For example, a space-saving arrangement of upper local coils on a patient is provided without changing a shape of the patient.

In accordance with one or more of the present embodiments, a magnetic resonance apparatus includes upper local coils embodied as inherently-stable. The upper local coils, in a state in which the upper local coils are connected to the connecting devices, are mechanically fixed to the patient couch and cover the patient in a dome-like shape. Electronics are arranged in the patient couch both in relation to lower local coils and also in relation to the upper local coils.

This enables separate holders for the upper local coils to be dispensed with, so that a space-saving arrangement may be realized. Because of the inherent stability of the upper local coils, a space remains provided between the upper local coils and the patient. The arrangement of the electronics also in relation to the upper local coils in the patient couch leads to relatively light upper local coils, so that the inherent stability may be provided relatively easily.

In one embodiment of the magnetic resonance apparatus, the couch surface is flat. This provides that the registration of the MR images is simplified and improved.

The upper local coils may, in relation to one upper local coil in each case, be connected alternatively on one side or both sides to the patient couch. The one-sided aspect or two-sided aspect may relate to the mechanical connection, to the electrical connection and to the electrical and mechanical connection.

In one embodiment of the magnetic resonance apparatus, the magnetic resonance apparatus also includes transmit and/or receive devices for transmitting and/or receiving ionizing radiation. In this case, the magnetic resonance apparatus may, for example, be embodied as a combined MR and PET apparatus.

In one embodiment, no sheath current filters and no flexible cables are arranged in the upper local coils. This enables the upper local coils to be built especially lightweight.

### BRIEF DESCRIPTION OF THE DRAWINGS

FIG. 1 shows one embodiment of a magnetic resonance apparatus in cross-section;

FIG. 2 shows a front view of the magnetic resonance apparatus from FIG. 1;

FIG. 3 shows one embodiment of a patient couch; and

FIGS. 4 and 5 show embodiments of upper local coils.

### DETAILED DESCRIPTION

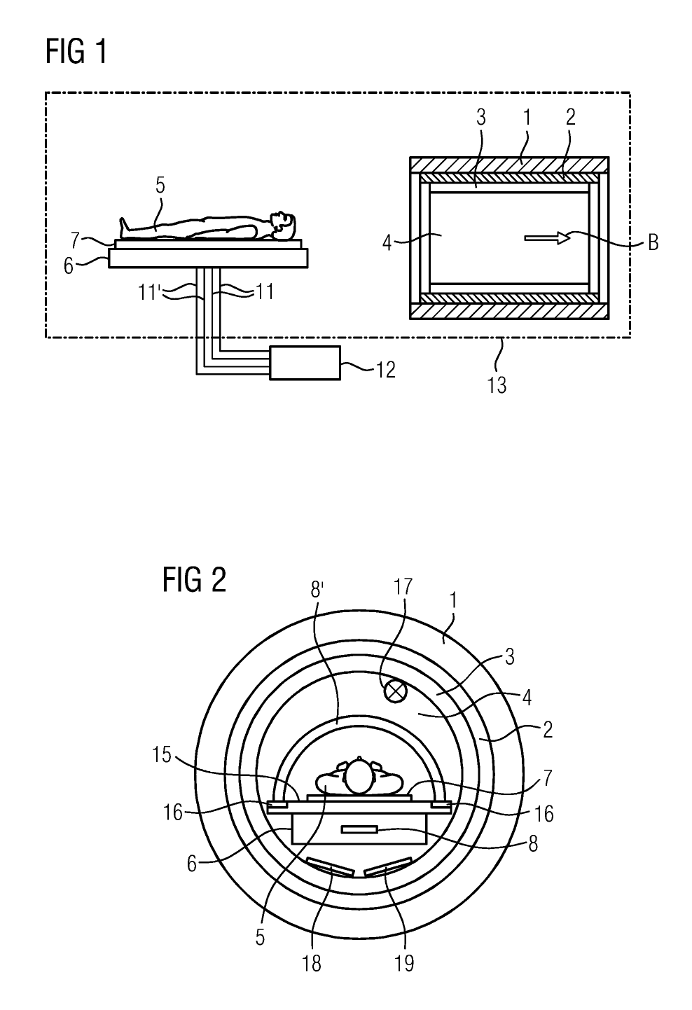

In accordance with FIGS. 1 and 2, one embodiment of a magnetic resonance apparatus includes a main magnet 1, a gradient magnet system 2 and a whole-body radio-frequency transmission arrangement 3. Using the main magnet 1, a temporally constant, locally homogeneous main magnetic field B is generated in an examination volume 4 of the magnetic resonance apparatus. Using the gradient magnet system 2, gradient fields may be overlaid on the main magnetic field B. Using the whole-body radio-frequency transmission arrangement 3, a patient 5 introduced into the examination volume 4 may be excited to transmit magnetic resonance signals.

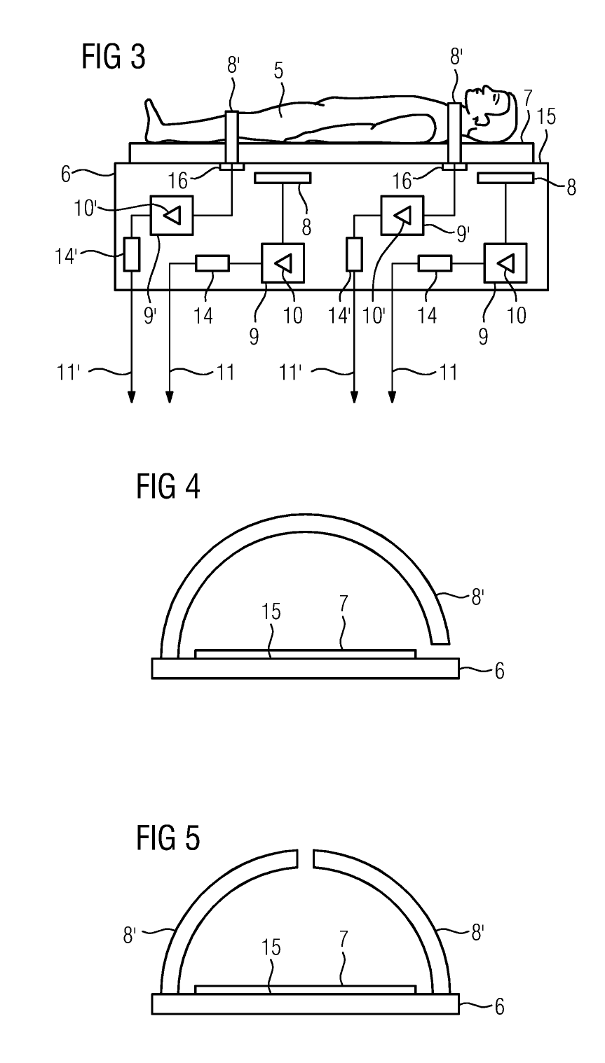

To introduce the patient 5 into the examination volume 4, the magnetic resonance apparatus includes a patient couch 6. The patient couch 6 has a couch surface 7. The patient 5 may be supported on the couch surface 7. Below the couch surface 7, in accordance with FIG. 3, local coils 8 are disposed in the patient couch 6. The local coils 8 are referred to below as lower local coils 8. The magnetic resonance signals excited in the patient 5 may be received by the lower local coils 8. The lower local coils 8 are connected to electronics 9 in each case. The electronics 9 include for the lower local coils 8 at least one pre-amplifier 10 in each case. If necessary, the electronics 9 may include further components such as frequency converters or AD converters, for example. The electronics 9 are disposed in the patient couch 6. The electronics 9 are connected via connecting lines 11 to an evaluation device 12. The evaluation device 12 is disposed outside the patient couch 6 (e.g., outside the examination room 13). Sheath current filters 14 may be arranged in the connecting lines 11, within or outside the patient couch 6.

The magnetic resonance apparatus also includes local coils 8′. The local coils 8′ are not arranged in the patient couch 6 but are attached above the patient couch 6 to the patient couch 6. The local coils 8′ are referred to below as upper local coils 8′. The magnetic resonance signals excited in the patient 5 may also be received by the upper local coils 8′.

On an upper side 15 of the patient couch 6, connecting devices 16 are arranged to sides of the patient couch 6. The connecting devices 16 are arranged at defined points of the patient couch 6. The upper local coils 8′ are detachably connected mechanically and electrically to the connecting devices 16.

The upper local coils 8′ are also connected to electronics 9′ in each case. Similarly to the electronics 9 of the lower local coils 8, the electronics 9′ for the upper local coils 8′ also include at least one pre-amplifier 10′ in each case. If necessary, similarly to the electronics 9 of the lower local coils 8, the electronics 9′ may include further components such as frequency converters or AD converters, for example. The electronics 9′ for the upper local coils 8′ are, however, not arranged on the upper local coils 8′. Like the electronics 9 of the lower local coils 8, the electronics 9′ are arranged in the patient couch 6. The electronics 9′ for the upper local coils 8′ are connected, like the electronics 9 for the lower local coils 8, to the evaluation device 12. Sheath current filters 14′ may likewise be arranged in the connecting lines 11′ within or outside the patient couch 6. In one embodiment, no sheath current filters are arranged in the upper local coils 8′.

The upper local coils 8′ are embodied inherently stable. The upper local coils 8′ may, however, be bent and elastically deformed by exerting external forces, but always assume a predefined shape again as soon as the external forces are no longer acting on the upper local coils 8′. The upper local coils 8′ may, for example, include a correspondingly stable-shape foam material, in which the antenna elements are embedded. The upper local coils 8′ are, provided the upper local coils 8′ are connected to the connecting devices 16, mechanically fixed to the patient couch 6. Because of the inherent stability of the upper local coils 8′ in conjunction with the defined arrangement of the connecting devices 16, the locations at which the upper local coils 8′ are arranged during the image acquisition are therefore uniquely defined. The pre-defined form of the upper local coils 8′ is selected such that the upper local coils 8′ in this state cover the patient 5 in the shape of a dome.

The support surface 7 may be flat. This embodiment is advantageous if the acquired MR images are to be used within the framework of the planning of a treatment of the patient 5 with ionizing radiation.

In accordance with the diagram in the embodiment of FIG. 2, the upper local coils 8′ are connected on both sides to the patient couch 6. As an alternative, the upper local coils 8′ in accordance with FIGS. 3 and 4 may be connected on just one side to the patient couch 6. In this case, the dome-type covering of the patient 5 in accordance with FIG. 4 may be realized by an individual, appropriately-dimensioned upper local coil 8′ that entirely covers the patient 5. As an alternative, the dome-like covering of the patient 5 in accordance with FIG. 5 may be realized by two appropriately-dimensioned local coils 8′ that cover the patient 5 from both sides in the shape of the dome.

In one embodiment, the magnetic resonance apparatus—see FIG. 2—has additional transmit and/or receive devices 17, 18, 19 for transmitting and/or receiving ionizing radiation (e.g., an x-ray source 17 and an x-ray detector 18 and/or a PET detector 19). In this case, both the image acquisition (in its entirety) and also the treatment with ionizing radiation may be carried out in the same apparatus (e.g., the corresponding magnetic resonance apparatus).

The present embodiments have many advantages. For example, the patient 5 may be covered by the local coils 8, 8′ in a space-saving manner both above and also below, without having to apply the upper local coils 8′ directly to the patient 5. Despite this, the upper local coils 8′ may still be built lightweight and relatively small, because exclusively antenna elements are to be arranged in the upper local coils 8′ but not other electronic devices. Disruptive cables emanating from the upper local coils 8′ may be avoided. For example, in combination with images captured by ionizing radiation, the arrangement of the electronics 9′ for the upper local coils 8′ in the patient couch 6 is advantageous, since through this, the location of the electronics 9′ is known in advance. Correction of any areas concealed by the electronics 9′ may therefore readily be provided.

Although the invention has been illustrated and described in detail by the exemplary embodiments, the invention is not restricted by the disclosed examples, and other variations may be derived therefrom by the person skilled in the art, without departing from the scope of protection of the invention.

It is to be understood that the elements and features recited in the appended claims may be combined in different ways to produce new claims that likewise fall within the scope of the present invention. Thus, whereas the dependent claims appended below depend from only a single independent or dependent claim, it is to be understood that these dependent claims can, alternatively, be made to depend in the alternative from any preceding or following claim, whether independent or dependent, and that such new combinations are to be understood as forming a part of the present specification.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. A magnetic resonance apparatus comprising:
a patient couch, wherein the patient couch has a couch surface on which a patient is supportable;
lower local coils operable to receive excited magnetic resonance signals of the patient, wherein the lower local coils are arranged in the patient couch, below the couch surface;
connecting devices arranged to sides on an upper side of the patient couch;
upper local coils, wherein the upper local coils are detachably connected mechanically and electrically to the connecting devices,
wherein the lower local coils and the upper local coils are connected to respective electronics,
wherein the electronics include at least one pre-amplifier in each case, the pre-amplifiers being connected via connecting lines to an evaluation device arranged outside the patient couch,
wherein the upper local coils, in a state in which the upper local coils are connected to the connecting devices, are fixed mechanically to the patient couch and cover the patient in a shape of a dome, and
wherein the electronics are arranged in the patient couch both in relation to the lower local coils and also in relation to the upper local coils.

2. The magnetic resonance apparatus of claim 1, wherein the couch surface is flat.

3. The magnetic resonance apparatus of claim 1, wherein the upper local coils are connected on one side or on both sides to the patient couch.

4. The magnetic resonance apparatus of claim 2, wherein the upper local coils are connected on one side or on both sides to the patient couch.

5. The magnetic resonance apparatus of claim 1, further comprising transmit, receive, or transmit and receive devices for transmitting, receiving, or transmitting and receiving ionizing radiation.

6. The magnetic resonance apparatus of claim 2, further comprising transmit, receive, or transmit and receive devices for transmitting, receiving, or transmitting and receiving ionizing radiation.

7. The magnetic resonance apparatus of claim 3, further comprising transmit, receive, or transmit and receive devices for transmitting, receiving, or transmitting and receiving ionizing radiation.

8. The magnetic resonance apparatus of claim 1, wherein no sheath current filters and no flexible cables are arranged in the upper local coils.

9. The magnetic resonance apparatus of claim 2, wherein no sheath current filters and no flexible cables are arranged in the upper local coils.

10. The magnetic resonance apparatus of claim 3, wherein no sheath current filters and no flexible cables are arranged in the upper local coils.

11. The magnetic resonance apparatus of claim 5, wherein no sheath current filters and no flexible cables are arranged in the upper local coils.

12. The magnetic resonance apparatus of claim 1, wherein the upper local coils are stable.
